(19) United States
(12) Patent Application Publication
(10) Pub. No.: US 2020/0330979 A1
(43) Pub. Date: Oct. 22, 2020

(54) DEVICES, KITS, AND METHODS FOR MONITORING DISEASE STATES

(71) Applicant: VANDERBILT UNIVERSITY, Nashville, TN (US)

(72) Inventors: Kevin J. CYR, Houston, TX (US); Christina C. MARASCO, Nashville, TN (US); Jennifer COLBY, Nashville, TN (US)

(21) Appl. No.: 16/303,703

(22) PCT Filed: May 23, 2017
(86) PCT No.: PCT/US2017/034059

§ 371 Date: Nov. 21, 2018

Related U.S. Application Data
(60) Provisional application No. 62/340,188, filed on May 23, 2016.

(51) Int. Cl. B01L 3/00; G01N 11/06; G01N 33/558; G01N 33/72

(52) U.S. Cl. CPC B01L 3/5023; G01N 11/06; G01N 33/558; G01N 33/721; B01L 2200/148; B01L 2300/0825; B01L 2300/069; B01L 2300/12; G01N 2800/22

(57) ABSTRACT

Various implementations include devices, kits, and methods for diagnosing and monitoring disease states using rheological properties of a bodily fluid within a lateral flow membrane. The devices, kits, and methods enable rapid diagnosis and management of those diseases that alter the physical properties of bodily fluids within an in vivo context. The ability to rapidly diagnose and manage those diseases enables primary care providers to provide detailed interventions at the point of care.

### CROSS REFERENCE TO RELATED APPLICATIONS

This application claims priority to U.S. Patent Application No. 62/340,188, entitled “Devices, Kits, and Methods for Monitoring Disease States,” filed May 23, 2016, the content of which is incorporated by reference in its entirety.

### BACKGROUND

Lateral flow diagnostic devices have recently gained interest for their implementation in point of care (PoC) settings to rapidly aid health care providers in diagnosing various health conditions. Lateral flow devices currently use a variety of methods to diagnose various conditions, including immunochromatographic ELISA assays. The most well-known lateral flow device, the pregnancy test, uses such methods. However, these devices have not been used to monitor rheological properties of bodily fluid for the purposes of diagnosing and monitor disease states related to the rheological properties of the bodily fluid.

Therefore, there is a need in the art for a diagnostic device for and methods of monitoring certain disease states that alter the rheological properties of a bodily fluid.

### BRIEF SUMMARY

Various implementations include devices, kits, and methods for diagnosing and monitoring disease states using rheological properties of a bodily fluid within a lateral flow membrane. The devices, kits, and methods enable rapid diagnosis and management of those diseases that alter the physical properties of bodily fluids within an in vivo context. The ability to rapidly diagnose and manage those diseases enables primary care providers to provide detailed interventions at the point of care.

Some implementations include a device for monitoring a disease state. The disease state is one that alters one or more rheological properties of a bodily fluid. The device includes a housing and a lateral flow strip. The housing defines a hollow interior portion and includes a first end and a second end spaced apart from and opposite the first end. The housing defines an inlet opening and an analysis opening. The inlet opening is adjacent the first end of the housing, and the analysis opening is disposed between the inlet opening and the second end of the housing. The lateral flow strip is disposed within the hollow interior portion. The lateral flow strip has a first end and a second end, and the first and second ends of the lateral flow strip are opposite and spaced apart from each other. The lateral flow strip includes an inlet area adjacent the first end of the lateral flow strip, an absorbent area adjacent the second end of the lateral flow strip, and an analysis area disposed between the inlet area and the absorbent area. The inlet area is disposed below the inlet opening for receiving bodily fluid through the inlet opening, and at least a portion of the analysis area is visible through the analysis opening of the housing. The lateral flow strip also comprises a reducing buffer solution. An observable rheological property of the bodily fluid is comparable to an expected rheological property to identify whether a disease state is present. The expected rheological property of the bodily fluid across the analysis area is associated with a healthy state.

Other implementations include a method of diagnosing a disease state by evaluating one or more rheological properties of a bodily fluid. The method includes: (1) depositing a sample of bodily fluid to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area, (2) comparing an observable rheological property of the bodily fluid with an expected rheological property associated with the bodily fluid in a healthy state, the observable rheological property comprising the flow of the bodily fluid from the inlet area toward the analysis area, and (3) diagnosing a disease state if the observable rheological property does not meet the expected rheological property, wherein the lateral flow strip comprises a reducing buffer solution.

Other implementations include a method of displaying a bodily fluid in a rheological property context. The method includes: (1) depositing a sample of bodily fluid to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area, and (2) allowing the bodily fluid to migrate through the lateral flow strip from the inlet area towards the analysis area for a defined period of time, wherein the defined period of time is the time a reference fluid having a rheological property takes to migrate from the inlet area to a point in the analysis area, and wherein the location of the bodily fluid relative to the analysis area after the defined period of time displays the bodily fluid in a rheological property context.

Other implementations include a test kit for monitoring a disease state that alters one or more rheological properties of a bodily fluid. The test kit includes a testing device, which includes a housing and a lateral flow strip. The testing device includes a housing that defines a hollow interior portion. The housing includes a first end and a second end spaced apart from and opposite the first end. The housing also defines an inlet opening and an analysis opening. The inlet opening is adjacent the first end of the housing, and the analysis opening is between the inlet opening and the second end of the housing. The lateral flow strip is disposable within the hollow interior portion. The lateral flow strip has a first end and a second end, and the first and second ends of the lateral flow strip are opposite and spaced apart from each other. The lateral flow strip includes an inlet area adjacent the first end of the lateral flow strip, an absorbent area adjacent the second end of the lateral flow strip, and an analysis area disposed between the inlet area and the absorbent area. The inlet area is disposable below the inlet opening for receiving bodily fluid through the inlet opening, and at least a portion of the analysis area is visible through the analysis opening. The lateral flow strip includes a reducing buffer solution. The bodily fluid being monitored has an expected rheological property across the analysis area that is associated with a healthy state, and an observable rheological property of the bodily fluid is comparable to the expected rheological property to identify whether a disease state is present.

Other implementations include a method of diagnosing a blood disorder disease that includes: (1) providing a sample of blood to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area; (2) capturing, via an image sensor, an image of the analysis area of the lateral flow strip within a field of view of the image sensor and electrically communicating image data associated with the captured image to a computer processor; (3) calculating, with the computer processor, a signal to noise ratio (SNR) based on the image data, the computer processor being in electrical communication with a memory, the memory storing instructions executable by the computer processor; and (4) identifying, by the computer processor, a biomarker density associated with the calculated SNR.

Other implementations include a system for diagnosing a blood disorder disease by evaluating one or more rheological properties of a sample of blood. The system includes a lateral flow strip, an image sensor, and a computer processor. The lateral flow strip has a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area, wherein an inlet area of the lateral flow strip is configured for receiving the blood sample. The image sensor is for capturing an image of the analysis area of the lateral flow strip. And the computer processor is in electrical communication with the image sensor and a memory. The memory stores instructions executable by the processor that cause the processor to: (1) receive image data associated with the image captured by the image sensor; (2) calculate a signal to noise ratio (SNR) from the image data; and (3) identify a biomarker density associated with the calculated SNR.

### BRIEF DESCRIPTION OF THE DRAWINGS

Various other objects, features and attendant advantages of various implementations will become fully appreciated as the same becomes better understood when considered in conjunction with the accompanying drawings, in which like reference characters designate the same or similar parts throughout the several views.

FIG. 1 is an exploded view of a rheological flow assay device according to one implementation.

FIG. 2 is an illustration of the introduction of a fluidic sample to the rheological flow assay device shown in FIG. 1.

FIG. 3 is a chart showing exemplary distances that a fluid may flow across the lateral flow strip over time.

FIG. 4A is a perspective view of a top portion and a bottom portion of a housing of a rheological flow assay device according to another implementation.

FIG. 4B is a top view of the device shown in FIG. 4A.

FIG. 4C is a side view of the device shown in FIG. 4A.

FIG. 5 is an illustration of a rheological flow assay device according to another implementation, showing the assay device from sample input to result and diagnostic output for sickle cell disease diagnosis, according to one implementation.

FIG. 6A shows the accuracy and sensitivity of the diagnostic test in the detection of sickle cell disease state as compared to known tests, such as high-performance liquid chromatography and solubility assay, according to one implementation.

FIGS. 6B and 6C illustrates detailed receiver operating characteristic (ROC) curves of the performance of the diagnostic test indicating cutoff thresholds, according to one implementation.

FIG. 7 illustrates exemplary steps of diagnosing a disease state using a kit according to various implementations.

FIG. 8 illustrates a method of diagnosing a disease state according to one implementation.

FIG. 9 illustrates a quantitative analysis of Hemoglobin S (Hb S) using a mobile computing device, such as a smartphone, according to one implementation.

FIG. 10 illustrates a system for collecting an image of the diagnostic device using a mobile computing device, according to one implementation.

FIG. 11A illustrates exemplary accumulation of samples on multiple test strips, wherein each sample has a different percentage of Hb S molecules. The SNR from the image data from images of each of the test strips is plotted against the known Hb S % to generate the exemplary calibration curve shown in FIG. 118.

FIGS. 12A and 12B illustrate a comparison of visual and mobile computing device based assessments of sickle cell disease, according to one implementation.

FIG. 13 illustrates a Bland-Altman plot comparing quantitative measurements of Hb S using a mobile computing device and using a high performance liquid chromatography (HPLC) analysis, according to one implementation.

FIGS. 14A and 14B illustrate linear regression and receiver operator characteristics for quantitative analysis, according to one implementation.

FIG. 15 illustrates correlations between average signal-to-noise (SNR) ratio and potential confounding factors, according to one implementation.

FIG. 16 illustrates a block diagram of a rheological property analysis computing system according to one implementation.

### DETAILED DESCRIPTION

Various implementations include devices, kits, and methods for enabling rapid monitoring (e.g., detection and on-going monitoring) of disease states that alter rheological properties of a bodily fluid. For example, sickle cell disease, which alters the rheological properties of blood, may be monitored. Other exemplary blood disorder disease states that may be monitored include coagulopathy, venous thrombosis, hyper IgM syndrome, Waldenstrom macroglobulinemia, primary amyloidosis, multiple myeloma, chronic lymphocytic leukemia, polycythemia, cryoglobulinemia, hemoglobinopathy, thalassemia, and compound heterozygous sickle cell diseases. In addition, other disease states that may be monitored include coronary artery disease, diabetes, rheumatoid arthritis, and dyslipidemia.

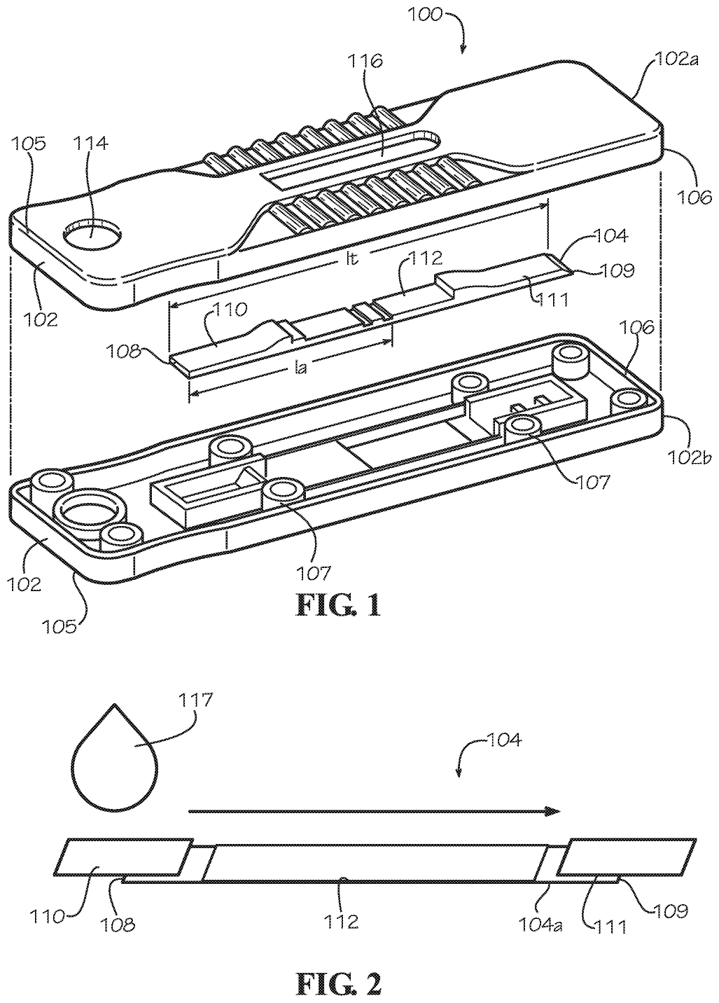

FIG. 1 illustrates an exploded view of an exemplary device 100 for monitoring a disease state that alters one or more rheological properties of a bodily fluid, such as those listed above, according to one implementation. The device 100 includes a housing 102, or cassette, and a lateral flow strip 104. The housing 102 includes an upper portion 102a and a lower portion 102b. Each portion 102a, 102b has a first end 105 and a second end 106. The upper portion 102a and lower portion 102b couple together and form a hollow interior portion. For example, the upper portion 102a may include posts (not shown) and the lower portion 102b may include bosses 107 for receiving the posts, or vice versa. The upper 102a and lower portions 102b may be secured together by frictionally engaging the posts within the bosses 107, for example. In other implementations (not shown), the portions 102a, 102b may be coupled together using other suitable fastening mechanisms, such as adhesives, screws, rivets, etc.

The housing 102 defines an inlet opening 114 and an analysis opening 116. The inlet opening is adjacent the first end 105 of the housing 102, and the analysis opening 116 is disposed between the inlet opening 114 and the second end 106 of the housing 102. In the implementation shown in FIG. 1, the inlet opening 114 and the analysis opening 116 are defined on the upper portion 102a of the housing 102. However, in other implementations (not shown), the inlet opening and/or the analysis opening may be are defined on lower portion 102b of the housing 102.

The lateral flow strip 104 comprises cellulose material, such as a paper lateral flow strip. In other examples, the lateral flow strip 104 comprises cellulose acetate, nitrocellulose, polyether sulfone; polyethylene, polypropylene, nylon, polyvinylidene fluoride (PVDF), polyester, silica, inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer, cloth, both naturally occurring (e.g., cotton) and synthetic (e.g., nylon or rayon), porous gels, such as silica gel, agarose, dextran, and gelatin; polymeric films, such as polyacrylamide; and so forth. In addition, at least a portion of the lateral flow strip 104 may be pretreated with a reducing buffer solution. Exemplary reducing buffer solutions include, but are not limited to any inorganic reducing salt in a buffer solution, such as sodium dithionate, sodium metabisulfite, potassium metabisulfite, potassium dithionite, and citrate.

The lateral flow strip 104 includes first end 108 and second end 109. An inlet area 110 of the lateral flow strip 104 is adjacent the first end 108 of the lateral flow strip 104, an absorbent area 111 of the lateral flow strip 104 is adjacent the second end 109 of the lateral flow strip 104, and an analysis area 112 is disposed between the inlet area 110 and the absorbent area 111. The analysis area 112 may extend between the inlet area 110 and the absorbent area 111. The inlet area 110, absorbent area 111, and analysis area 112 may be formed from one strip of cellulose material, or one or more of the areas 110, 111, 112 may be formed separately and coupled to a base or substrate and in fluid communication with each other. For example, FIG. 2 illustrates an implementation in which the lateral flow strip 104 includes a base 104a, a first piece of material coupled to the base 104a adjacent the first end 108 to form the inlet area 110, and a second piece of material coupled to the base 104a adjacent the second end 109 to form the absorbent area 111. The analysis area 112 is part of the base 104a. In one implementation, the first piece of material forming the inlet area 110 may include a cellulose fiber, and the second piece of material forming the absorbent area 111 may include nitrocellulose or alpha cellulose (e.g., Millipore chromatography paper). Other materials may be selected for the various areas 110, 111, 112 depending on the rheological properties of the bodily fluid to be tested. For example, one of more areas of the lateral flow strip 104 may include glass fiber and/or cotton.

In some implementations, the lateral flow strip 104 has a ratio of a length between a portion of the analysis area 112 and the inlet area 110, referred to hereinafter as Iₐ, to a total length of the strip 104, referred to hereinafter as Iₜ, of 1:5. For example, if the expected rheological property is for the bodily fluid to flow from the inlet area 110 to a portion of the analysis area that is about 14 to about 16 mm away from the inlet area 110 within the predetermined time window, the total length of the lateral flow strip 104 may be about 70 to about 80 mm long. In addition, the lateral flow strip may be about 1 to about 2 mm high and about 5 mm wide, according to some implementations. The Iₐ:Iₜ ratio of the lateral flow strip may change for each disease state depending on how the rheological profile is altered within the given disease state. And, the rheological profile may depend on the dimensions of the lateral flow strip and the materials used for the lateral flow strip.

The lateral flow strip 104 is disposed between the upper portion 102a and the lower portion 102b of the housing 102 within the hollow interior portion. The inlet area 110 is disposed below the inlet opening 114, and at least a portion of the analysis area 112 is disposed below the analysis opening 116.

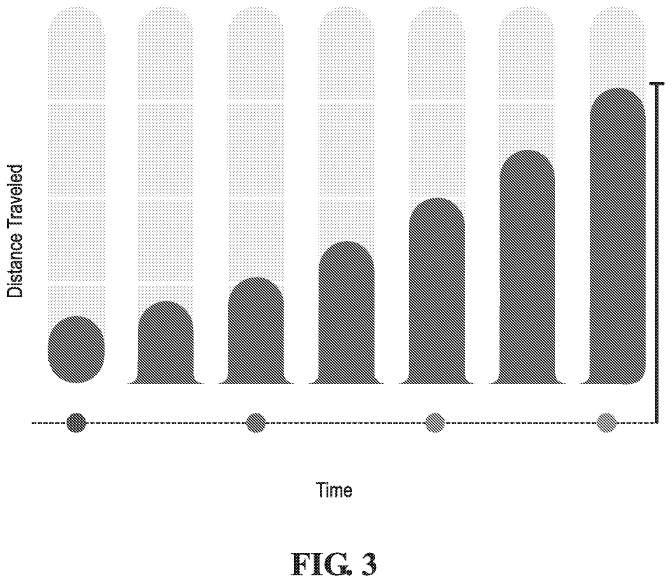

A bodily fluid 117 to be evaluated with the device 100 is received through the inlet opening 114. The bodily fluid is deposited on the inlet area 110 and passively flows across the lateral flow strip 104 from the inlet area 110 through the analysis area 112 and toward the absorbent area 111. An observable rheological property of the bodily fluid is comparable to an expected rheological property of the bodily fluid to identify whether a disease state is present. The expected rheological property of the bodily fluid across the analysis area is associated with a healthy state. The expected rheological property of the bodily fluid may include viscosity, flow rate (minimum or maximum, for example), shear rate, or shear stress, for example. For example, FIG. 3 illustrates a chart of the expected flow of the bodily fluid across the lateral flow strip 104 over time. The flow of the bodily fluid being tested may be compared to this chart to identify the disease state. Alternatively or in addition, the distance traveled by the bodily fluid being tested within a particular time period (e.g., 5 minutes) may be compared to the expected distanced traveled by the bodily fluid within that particular time period.

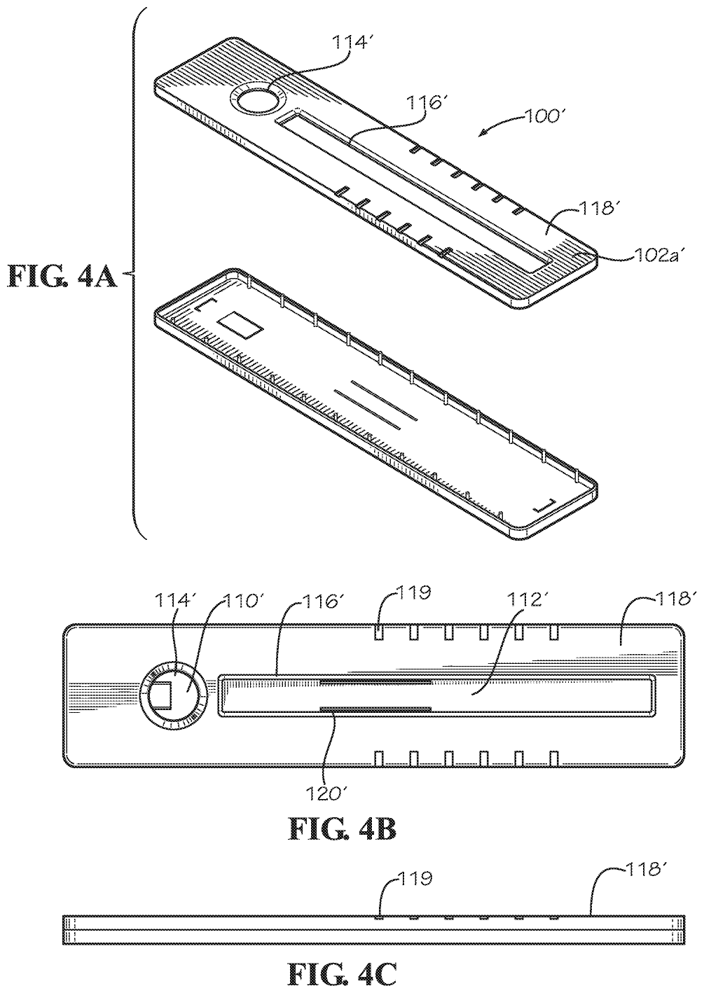

In the implementation shown in FIGS. 4A through 4C, an outer surface 118′ of the upper housing 102a′ includes visible marks 119′ adjacent the analysis opening 116′. The marks may be associated with a particular distance from the inlet area 112′. Thus, the health care provider can compare the distance traveled by the bodily fluid to the distance expected for healthy bodily fluid. The distance traveled may be associated with a particular time window during which the fluid flows before being evaluated (e.g., a minimum or maximum flow rate). The marks 119′ may be printed on the outer surface 118′ using an ink or other suitable material and/or the marks 119′ may be recessed into or extend away from the outer surface 118′. In addition, the lateral flow strip 104′ may include one or more marks 120′ adjacent the analysis area 112′ that assist a health care provider with comparing the rheological properties of the bodily fluid with the expected rheological property of the fluid to identify whether a disease state is present.

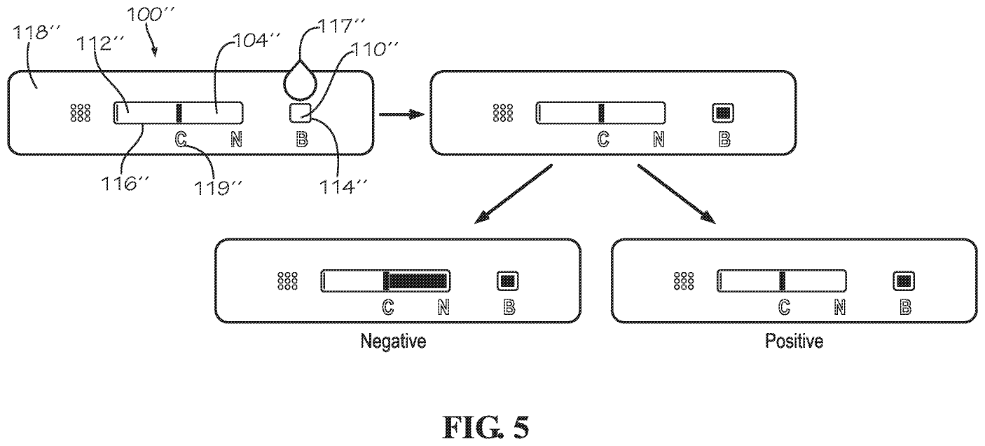

FIG. 5 illustrates another implementation of the device 100″ and the exemplary flow of bodily fluid through the lateral flow strip 104″ based on the presence or absence of the disease state. The device 100″ includes housing 102″ that defines input opening 114″ and analysis opening 116″. Input area 110″ of the lateral flow strip 104″ is disposed below the input opening 114″, and the analysis area 112″ is disposed below the analysis opening 116″. Bodily fluid 117″ is deposited through the input opening 114″ onto input area 110″ of the lateral flow strip 104″ and flows toward the analysis area 112″ of lateral flow strip 104″. The outer surface 118″ of the housing 102″ includes marks 119″ that indicate a distance to which the bodily fluid is expected to flow within a predetermined time window. As shown in FIG. 5, the distance that the bodily fluid has traveled after being deposited on the input area 110″ is compared to the expected distance traveled for healthy bodily fluid. The device 100″ on the left shows flow of the bodily fluid to the mark “C” on the outer surface 118″, and the device 100″ on the right shows flow that does not reach the mark “C”. In this example, presence of the disease state is associated with flow that does not reach the mark “C” within the predetermined time window.

These devices 100, 100′, 100″ may be useful for monitoring (e.g., detection and on-going monitoring) sickle cell disease. For example, the devices 100, 100′, 100″ may be useful for monitoring hydroxyurea therapy. Sickle cell disease is a genetic defect that results in a phenotypic change in red blood cell morphology. Sickled red blood cells form a distinct crescent shape that may lead to hemoglobin polymerization, erythrocyte stiffening, and subsequent vaso-occlusion. Because blood from a person having sickle cell disease is more viscous than blood from a healthy subject, the blood affected by sickle cell disease does not flow as far (or as fast) across the lateral flow strip 104, 104′, 104″ as blood from healthy subjects. For example, in an implementation in which the disease state to be evaluated is sickle cell disease, the bodily fluid is blood, and the expected rheological property is minimum flow rate, sickle cell disease is identified if the flow rate of the blood from the input area toward the analysis area is less than the minimum flow rate. For example, the minimum flow rate may be around 0.75 mm²/s, and blood having sickle cell disease may have a flow rate of about 0.60 mm²/s. Thus, if the blood does not travel at least 0.75 mm²/s, the blood may be identified as having sickle cell disease.

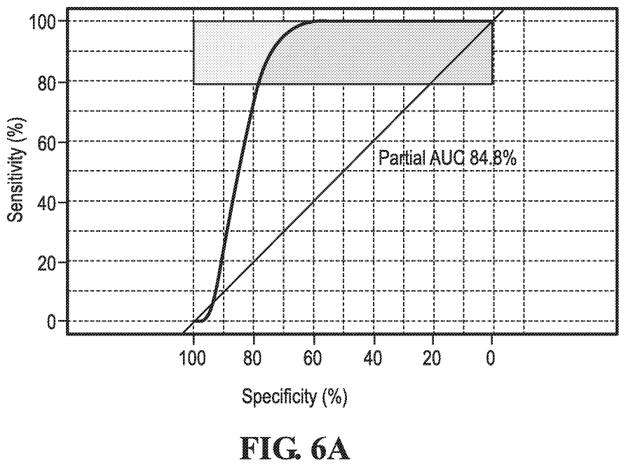

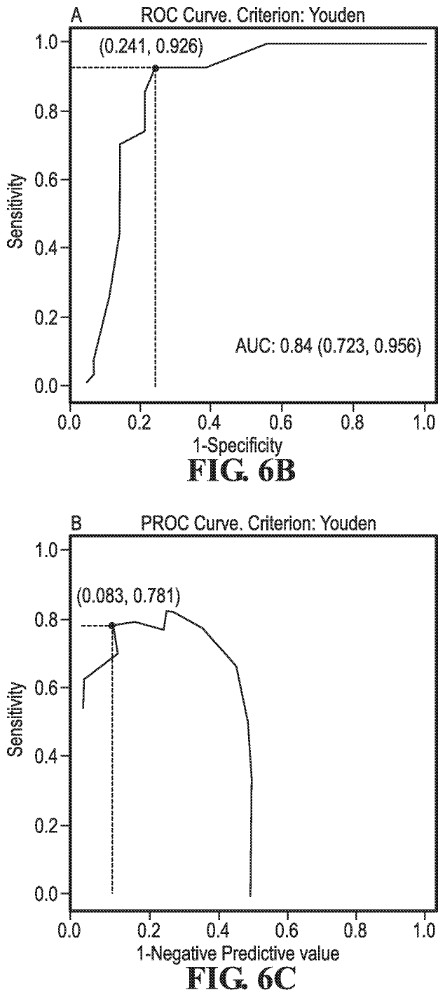

FIG. 6A illustrates the high performance of the diagnostic test in assessing sickle cell disease states compared to known tests. The Area Under the Curve (AUC) is very high and demonstrates the accuracy in detecting the disease. FIGS. 6B and 6C indicate the cutoff points for diagnostic determination as calculated by the Youden Index. The optimal cutoff points to obtain maximum sensitivity and specificity are illustrated in FIG. 68, and the optimum Positive Predictive Value and Negative Predictive Value are shown in FIG. 6C.

As another example, the device 100, 100′, 100″ may be useful for monitoring coagulopathy, which is a condition in which the blood has lost the ability to coagulate. In such an example, the bodily fluid is blood and the expected rheological property is maximum flow rate. Coagulopathy is identified if the flow rate of the blood from the input area toward the analysis area is more than a maximum expected flow rate.

In various implementations, the testing is performed in room temperature conditions at a point of care facility or in a laboratory.

Furthermore, the devices 100, 100′, 100″ may be used to monitor disease states by comparing the density of the color of the bodily fluid across the lateral flow strip 104, 104′, 104″. A unique precipitate may form on the lateral flow strip 104, 104′, 104″ in an indication that may yield a diagnostic result. Essentially, the rheological flow may cause the accumulation and depositing of the precipitate, which may be used itself as an indicator. In this way, the capacity to detect a disease state is provided that is not just based on the distance traveled but on the density of the color or on the distance-mediated accumulation of the blood cells on the lateral flow strip.

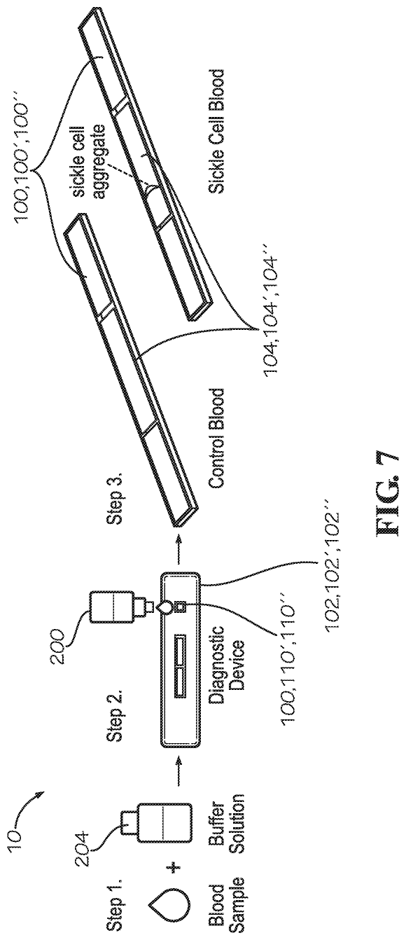

FIG. 7 shows one example of using a lateral flow strip, such as lateral flow strip 104, 104′, 104″, with blood to diagnose sickle cell disease by observing the color density of the bodily fluid. In this example, sickle cell disease is diagnosed in response to observing an accumulation of the blood in the analysis area that is not present in non-sickle cell blood. The accumulation results in a higher color density. The color density may be correlated to the percentage of a given element in a sample. For example, in sickle cell disease, that value is correlated to hemoglobin S (Hb S) concentration. The accumulation may be due, at least in part, to the polymerization within the buffer solution, which enables the sickle cells to fuse to one another and form a distinctly dark aggregate within a positive sample.

These devices 100, 100′, 100″ provide relatively quick results regarding the rheological properties of the bodily fluid tested, according to some implementations. Thus, devices 100, 100′, 100″ may be useful for rapidly screening bodily fluids from donors. For example, the devices 100, 100′, 100″ may be used to rapidly screen blood donors for blood abnormalities that are not wanted in donors.

The devices 100, 100′, 100″ described above may be provided with a kit, according to some implementations. In the implementation shown in FIG. 7, the kit 10 includes a housing, such as housings 102, 102′, 102″, a plurality of lateral flow strips, such as strips 104, 104′, 104″, and an applicator 200 for receiving the bodily fluid from a patient and depositing the bodily fluid on the inlet area, such as inlet area 110, 110′, 110″. The applicator 200 may include, for example, a pipette or a stick (e.g., a finger stick). The kit 10 may also include a container of calibration solution associated with the disease state and/or a container 204 of reducing buffer solution for mixing with the bodily fluid. The reducing buffer solution may be applied to the lateral flow strip ahead of depositing the bodily fluid onto the lateral flow strip such that the bodily fluid and reducing buffer solution mix on the lateral flow strip. Alternatively, the reducing buffer solution may be mixed in a separate container with the bodily fluid ahead of depositing the mixture onto the lateral flow strip.

As shown in Step 1 in FIG. 7, a fingerstick volume of blood (e.g., approximately 25 μL) is mixed with a sodium metabisulfite buffer solution (e.g., 100 μL) 204 in a micro centrifuge tube. In Step 2, the blood/buffer is inverted to mix and delivered to the inlet area 110, 110′, 110″ of the lateral flow strip 104, 104′, 104″ on the diagnostic device 100, 100′, 100″ using a disposable pipette 200. In Step 3, after five minutes, samples are analyzed visually. The distinct capillary flow pattern of sickle cell blood causes a noticeable aggregate to form on the lateral flow strip 104, 104′, 104″. Control HbA blood wicks across the lateral flow strip 104, 104′, 104″, and it does not form a distinct visible aggregate. Instead, the lateral flow strip 104, 104′, 104″ appears pale pink, which is shown as the “Control Blood” sample in FIG. 7. Positive test results may be rapidly visualized by the naked eye without the need for additional analytical equipment, which is shown in the “Sickle Cell Blood” sample shown in FIG. 7.

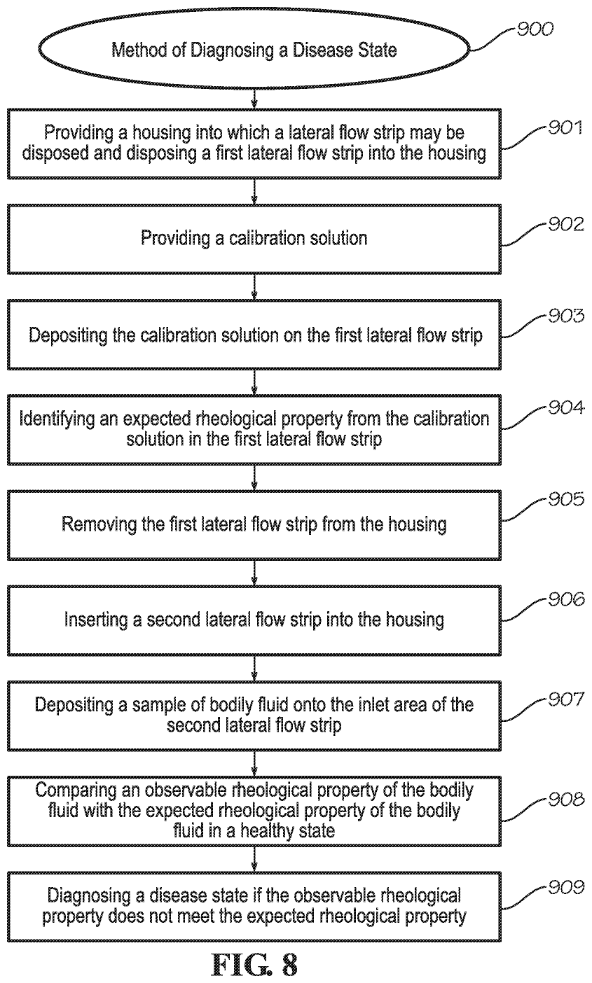

FIG. 8 illustrates a method of diagnosing a disease state by evaluating one or more rheological properties of a bodily fluid. The method 900 begins at step 901 by providing a housing, such as housings 102, 102′, 102″ described above, into which a lateral flow strip may be disposed and disposing a first lateral flow strip into the housing. Alternatively, the lateral flow strip may be pre-disposed in the provided housing. A calibration solution associated with the disease state is also provided, which is shown as step 902. The calibration solution is deposited onto the inlet area of the lateral flow strip, such as the inlet areas 110, 110′, 110″ and lateral flow strips 104, 104′, 104″ described above in relation to FIGS. 1, 2, 4A-4C, and 5, which is shown as step 903. The expected rheological property for the bodily fluid is identified based on the rheological property of the calibration solution, as shown in step 904. The first lateral flow strip is then removed from the housing, shown as step 905, and a second lateral flow strip is disposed within the housing, shown as step 906. Next, a sample of bodily fluid is deposited onto the inlet area of the second lateral flow strip, shown as step 907. Then, in step 908, an observable rheological property of the bodily fluid is compared with the expected rheological property associated with the bodily fluid in a healthy state. The observable rheological property includes the flow of the bodily fluid from the inlet area toward the analysis area. In step 909, a disease state is diagnosed if the observable rheological property does not meet the expected rheological property. In other implementations, steps 902-906 may not be necessary.

In some implementations, a mobile (e.g., handheld) computing device, such as a smartphone, may be used to evaluate the results of the rheological flow assays. For example, in some implementations, the mobile computing device may be used to capture an image of the analysis area, and image data associated with the image is evaluated with calibrated data to identify an Hb S % concentration.

In particular, in some implementations, the mobile computing device includes a computer processor in electrical communication with a memory and an image sensor, such as a camera, associated with the mobile computing device. The memory stores instructions that are executed by the processor. The instructions cause the processor to receive image data associated with an image of the analysis area of the lateral flow strip after the predetermined time window, compare at least a portion of the image data to expected image data, generate an evaluation of the comparison, and communicate the evaluation to a user, such as via the mobile computing device or other computing device. The processor may be on the mobile computing device or on a computing device that is remotely disposed from the mobile computing device. The image data and/or evaluation may also be communicated to a cloud based computing device that is remotely located from the mobile computing device and stored thereon and/or communicated further to another computing device.

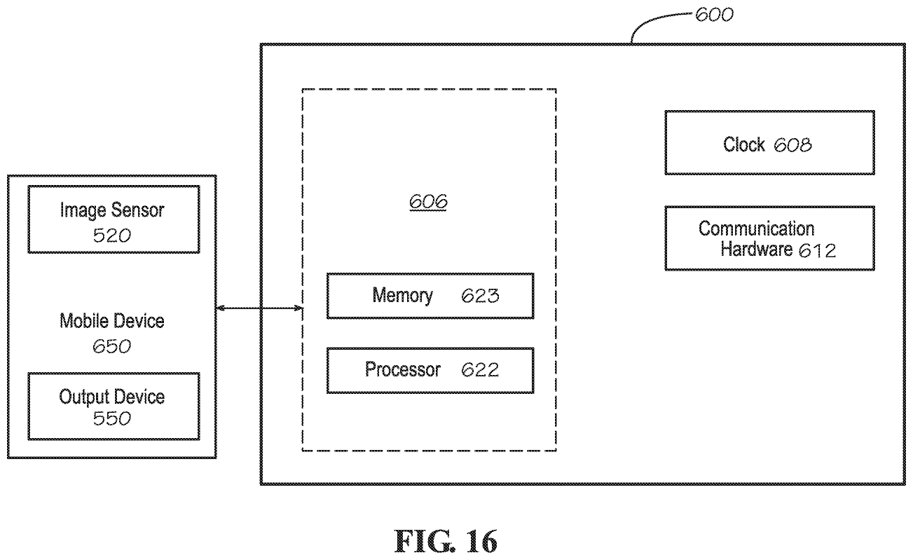

FIG. 16 illustrates a block diagram of a rheological property analysis computer system 600, according to one implementation. The system 600 includes a computing unit 606, a system clock 608, and communication hardware 612. In its most basic form, the computing unit 606 includes a processor 622 and a system memory 623. The processor 622 may be one or more standard programmable processors that perform arithmetic and logic operations necessary for operation of the system 600. The processor 622 may be configured to execute program code encoded in tangible, computer-readable media. For example, the processor 622 may execute program code stored in the system memory 623, which may be volatile or non-volatile memory. The memory 623, which can be embodied within non-transitory computer readable media, stores instructions for execution by the processor 622. The system memory 623 is only one example of tangible, computer-readable media. In one aspect, the computing unit 606 can be considered an integrated device, such as firmware. Other examples of tangible, computer-readable media include floppy disks, CD-ROMs, DVDs, hard drives, flash memory, or any other machine-readable storage media, wherein when the program code is loaded into and executed by a machine, such as the processors 622, 632, the machine becomes an apparatus for practicing the disclosed subject matter.

In addition, the processor 622 is in electrical communication with an image sensor (e.g., image sensor 520 described below). In some implementations, the system 600 further includes a transceiver that is in electrical communication with the processor 622 and a mobile (e.g., handheld) computing device 650 having an output device 550 (e.g., display screen, speaker).

In other implementations, the system 600 may include two or more processors and/or memories. In addition, in the implementation shown in FIG. 16, the processor and memory are disposed on a separate computing device that is remotely located from the mobile computing device 650 and in communication with the mobile computing device 650 over a wired or wireless network. However, in other implementations, the one or more components of system 600 may be disposed on the mobile computing device 650.

Aspects of the present invention are described above with reference to flowchart illustrations and/or block diagrams of methods, apparatus (systems) and computer program products according to implementations of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

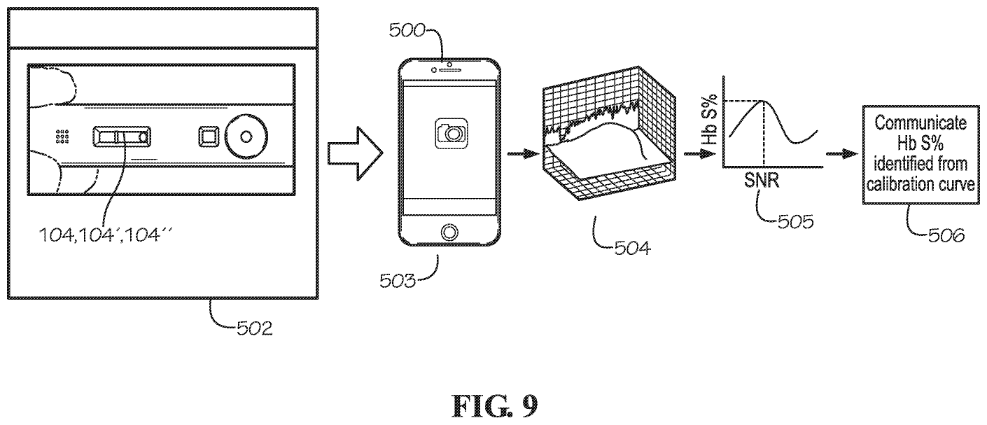

FIG. 9 illustrates steps for evaluating a sample according to one implementation. As shown in box 502 of FIG. 9, the lateral flow strip 104, 104′, 104″ may be visually inspected to determine if aggregates formed, indicating the tested sample is positive for sickle cell disease. Alternatively or in addition to a visual inspection, an image sensor (e.g., a camera on smartphone 500) can be used to capture an image of the lateral flow strip 104, 104′, 104″, as shown in step 503, and image data associated with this image is electrically communicated to and evaluated by a computer processor (e.g., computer processor 622) to determine the Hb S % or perform other evaluations of the sample that may not be possible from a visual inspection by the health care provider. In this implementation, the computer processor is remotely disposed from the mobile computing device. Thus, the mobile computing device communicates the image data to the remotely disposed computer processor, and the remotely disposed computer processor isolates the image data associated with the test region and calculates a signal-to-noise ratio (SNR), as shown in step 504. The calculation may be performed using ImageJ software, according to some implementations. The SNR is then compared to a standard curve of known Hb S % versus SNR, and the Hb S % present in the sample is identified, as shown in Step 505. The computer processor then communicates the identified HB 5% to the mobile computing device, as shown in Step 506.

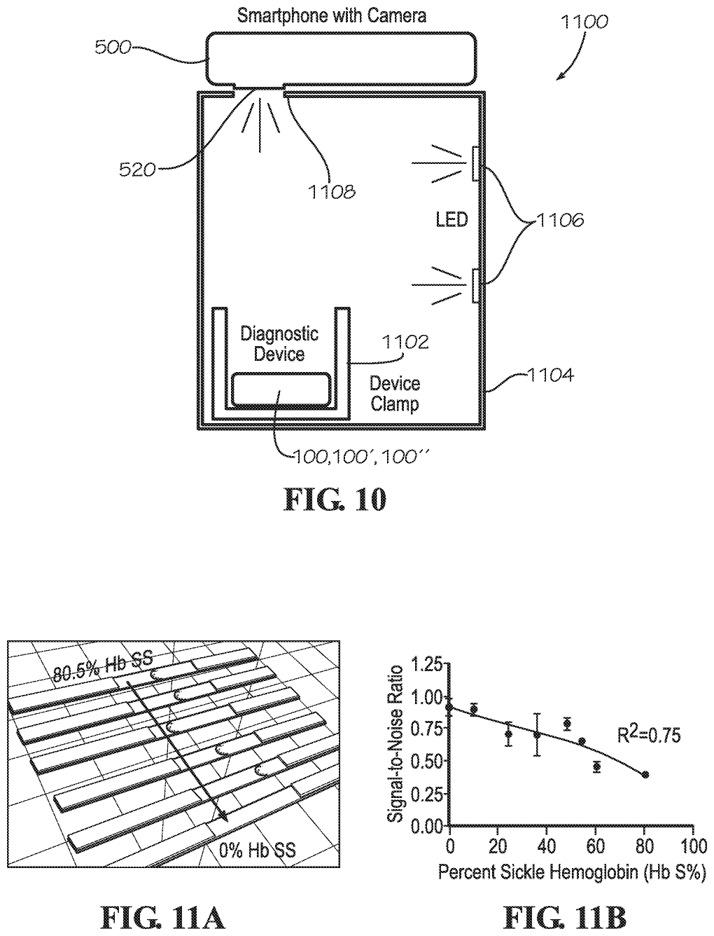

FIG. 10 illustrates an imaging system 1100 according to one implementation. The imaging system 1100 reduces the variability of imaging conditions and standardizes results. The diagnostic device 100, 100′, 100″ is disposed within a device clamp 1102 that is disposed within a box 1104 that is made from or includes a light blocking material (e.g., a laser-cut black acrylic box). An interior light source 1106 within the box, such as LEDs, uniformly illuminates the diagnostic device 100, 100′, 100′. The top of the box 1104 defines an opening 1108 through which an image sensor 520 of the mobile computing device (e.g., smartphone 500) captures images of the diagnostic device 100, 100′, 100″ disposed within the device clamp 1102 in the box 1104. The images captured are within a field of view of the image sensor 520. Image data associated with the captured images within the field of view of the image sensor 520 is collected and analyzed as described above. For example, the image data may be communicated on a frame by frame basis. In addition, according to some implementations, the image sensor 520 may include a two dimensional or three dimensional camera, for example.

FIG. 11A illustrates exemplary accumulation of samples on multiple test strips, wherein each sample has a different percentage of Hb S molecules. The SNR from the image data from images of each of the test strips is plotted against the known Hb S % to generate the exemplary calibration curve in FIG. 11B. In particular, subject samples, including those that were positive for sickle cell disease (Hb SS) and those that were negative (Hb A) for sickle cell disease, were mixed to generate standards with known relative sickle hemoglobin levels (Hb S %). Pooled samples of 80.5% Hb S blood were mixed with pooled samples of control blood in varying ratios from 0% to 100%. Individual samples were analyzed independently using three separate diagnostic devices, and the average signal-to-noise-ratio (SNR) was calculated to establish standard curve values. A polynomial curve was fit to the data (R²=0.75) so that quantification of Hb S % could be interpolated for future samples. The position of the aggregate in the test strips varied slightly from test to test due to the individual properties of the sample. The SNR agreed among the different tests, with a 95% C.I., although some increased variability was seen in samples with approximately 35-50 Hb S % levels. As noted above in relation to FIG. 9, this calibration curve may be used by the processor to identify Hb S % in imaged test strips.

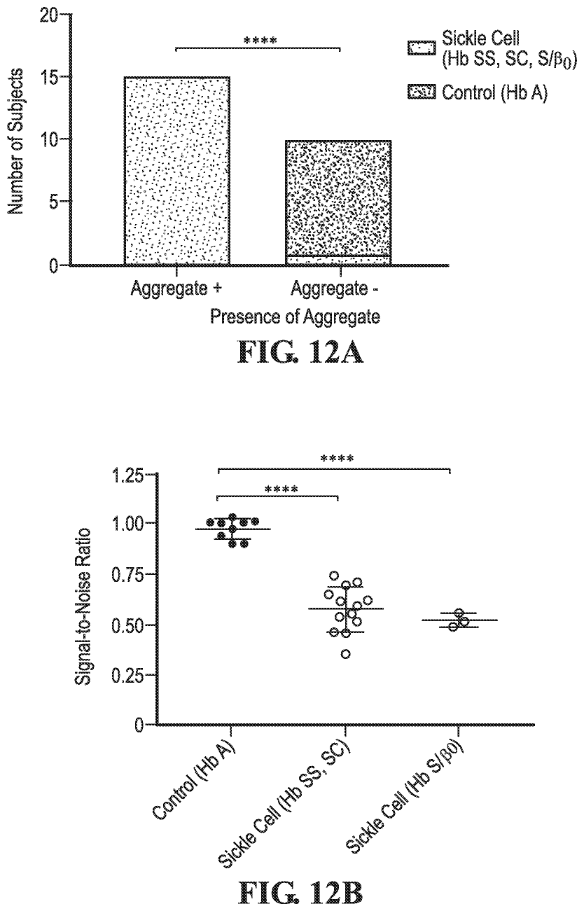

FIGS. 12A-12B illustrate that visual and smartphone-based assessments are sensitive to sickle cell disease. In FIG. 12A, visual confirmation of the presence of an aggregate was evaluated for use as a diagnostic indicator of sickle cell disease. A statistically significant difference (Fisher's Exact Test, p<0.0001) was seen between aggregate formation for sickle cell positive samples and control. In FIG. 12B, the image captured by the smartphone was analyzed and used to calculate the SNR for individuals with sickle cell disease (Hb SS and Hb S/β0 thalassemia). The value of the SNR shows a statistically significant difference between sickle cell disease and control subjects, highlighting the ability of the method to detect sickle cell disease (ANOVA, p<0.0001).

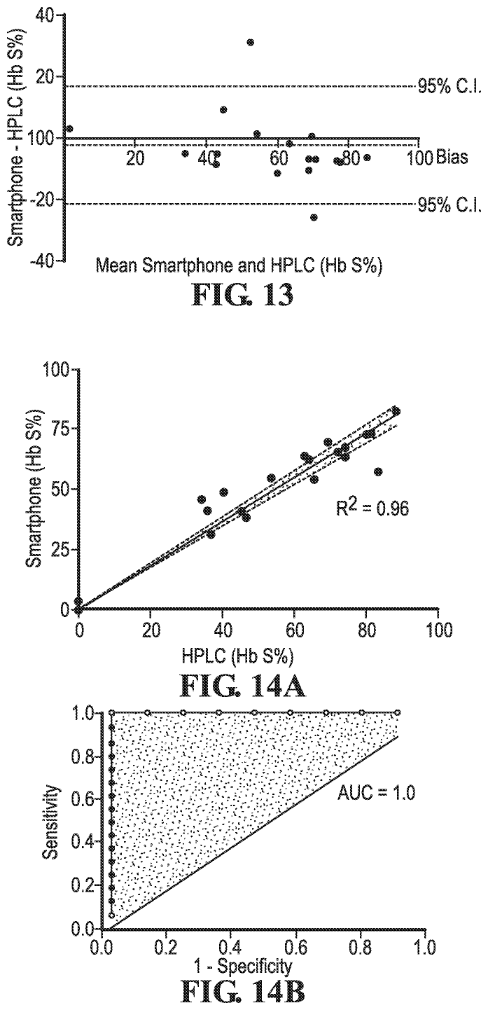

FIG. 13 illustrates that the smartphone analysis reported similar results in comparison to gold-standard high performance liquid chromatography (HPLC) analysis with an average error, or bias, of 2.27% (95% C.I. −21.3%-16.8%). The results cluster tightly around the x-axis, indicating the methods closely agree. Except for two samples, the results from analyzing the image captured by the smartphone agreed with HPLC results and may provide a quantitative indicator of Hb S % that can be used in the management of patients with sickle cell disease.

FIGS. 14A and 14B illustrate linear regression and receiver operating characteristics for quantitative analysis. In FIG. 14A, linear regression analysis indicated that the smartphone quantitative results agreed with gold-standard HPLC results (R²=0.96). In FIG. 14B, the receiver operating characteristics illustrated that the smartphone-based screening for sickle cell disease was accurate and can be tuned to set cutoff points for a positive diagnosis. At a SNR greater than 0.90, the sensitivity approached 1.0 and the specificity approached 0.89 with a likelihood ratio of 9:1, indicating a strong correspondence between diagnostic values and sickle cell disease. The SNR can be investigated in future studies with larger cohorts to evaluate effective thresholds for screening.

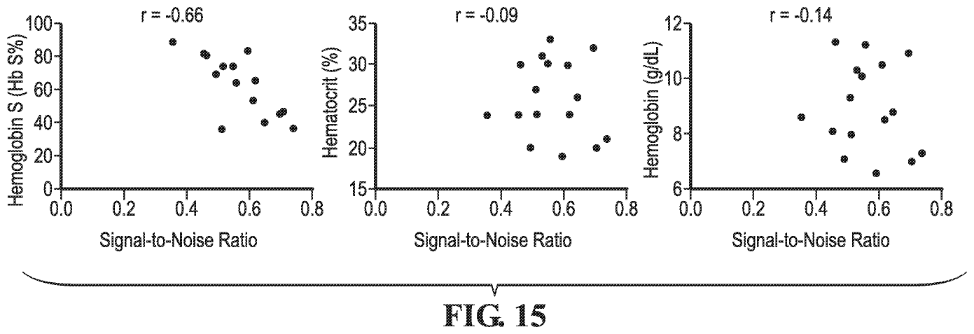

FIG. 15 illustrates the correlation between SNR and potential confounding factors. Confounding factors were analyzed using both correlation and multiple linear regression to investigate their effects on sample analysis. Hb S % had a strong and statistically significant inverse correlation with SNR as hypothesized (r=−0.66, p=0.0058). Neither hematocrit (HCT) nor total hemoglobin (HB) demonstrated a significant correlation with SNR, indicating that neither factor contributed to the diagnostic results.

These evaluations reduce the burden on health care providers to understand complex diagnostic tests and enables them to read, diagnose, and store information using the mobile computing device. In addition, the information from the evaluation may be communicated and integrated with existing electronic medical record (EMR) systems to store pertinent patient tests, thus reducing the time expended by health care providers to chart patients.

It is estimated that over 70% of deaths related to sickle cell disease may be eliminated through early diagnosis and treatment. However, current methods require expensive equipment, technical expertise, or cold storage reagents. For example, current methods include gel electrophoresis, microscope identification, or chemical solubility studies. In addition, the lack of knowledge about areas afflicted by sickle cell disease makes allocation of resources for testing and diagnosis a major challenge.

The devices, methods, and kits described herein utilize the unique design characteristics of lateral flow strips to analyze the rheological properties, or physical properties, of these bodily fluids to assess and monitor disease states. These devices, methods, and kits, which may be referred to generally as rheological flow assays, evaluate actual properties of fluids as they wick through the lateral flow strip. These rheological flow assays present the ability to diagnose and monitor the effect of diseases within the tissues of the body more accurately than immunochromatographic or chemical based assays, according to certain implementations. These rheological flow assays may be able to eliminate the expensive equipment, antibodies, manufacturing processes, cold storage reagents, and technical expertise typically associated with point of care diagnostics, which allows them to be used in lower resource settings. In addition, these rheological flow assays can provide information using microliters of bodily fluid. Furthermore, the assays may be used by relatively untrained individuals. These rheological flow assays may also be applicable to monitoring many disease states.

While the foregoing description and drawings represent the preferred implementation of the present invention, it will be understood that various additions, modifications, combinations and/or substitutions may be made therein without departing from the spirit and scope of the present invention as defined in the accompanying claims. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. In addition, features described herein may be used singularly or in combination with other features. The presently disclosed implementations are, therefore, to be considered in all respects as illustrative and not restrictive, the scope of the invention being indicated by the appended claims and not limited to the foregoing description.

1. A device for monitoring a disease state, the disease state altering one or more rheological properties of a bodily fluid, the device comprising:
a housing defining a hollow interior portion, the housing comprising a first end and a second end spaced apart from and opposite the first end, the housing defining an inlet opening, the inlet opening being adjacent the first end of the housing; and
a lateral flow strip disposed within the hollow interior portion, the lateral flow strip having a first end and a second end, the first and second ends of the lateral flow strip being opposite and spaced apart from each other, wherein the lateral flow strip comprises an inlet area adjacent the first end of the lateral flow strip, an absorbent area adjacent the second end of the lateral flow strip, and an analysis area disposed between the inlet area and the absorbent area, the inlet area being disposed below the inlet opening for receiving bodily fluid through the inlet opening,
wherein:
the housing defines an analysis opening through which at least a portion of the analysis area of the lateral flow strip is visible,
the lateral flow strip comprises a reducing buffer solution,
the bodily fluid has an expected rheological property across the analysis area that is associated with a healthy state, and
an observable rheological property of the bodily fluid is comparable to the expected rheological property to identify whether a disease state is present.

2. The device of claim 1, wherein the disease state is sickle cell disease, the bodily fluid is blood, the expected rheological property is a minimum flow rate of the blood across the analysis area, and sickle cell disease is identified if the flow rate of the blood from the input area toward the analysis area is less than the minimum flow rate.

3. The device of claim 1, wherein the disease state is coagulopathy, the bodily fluid is blood, the expected rheological property is a maximum flow rate of the blood across the analysis area, and coagulopathy is identified if the flow rate of the blood from the input area toward the analysis area is more than the maximum flow rate.

4. The device of any one of the preceding claims, wherein the reducing buffer solution comprises an inorganic reducing salt.

5. The device of anyone of the preceding claims, wherein the lateral flow strip comprises a cellulose material.

6. The device of any one of the preceding claims, wherein the inlet area and the absorbent area comprise a cellulose fiber, and the analysis area comprises nitrocellulose.

7. The device of any one of claims 1 through 4, wherein the lateral flow strip comprises a glass fiber material.

8. The device of any one of claims 1 through 4, wherein the lateral flow strip comprises a cotton material.

9. The device of claim 1, wherein the disease state is selected from one of the following: sickle cell disease, dyslipidemia, coagulopathy, venous thrombosis, hemoglobinopathy, thalassemia, and compound heterozygous sickle cell diseases.

10. The device of claim 1, wherein the disease state is selected from one of the following: hyper IgM syndrome, Waldenstrom macroglobulinemia, primary amyloidosis, multiple myeloma, chronic lymphocytic leukemia, polycythemia, and cryoglobulinemia.

11. The device of any one of the preceding claims, wherein the first surface of the housing further comprises a mark adjacent the analysis opening, the mark indicating an expected distance for the bodily fluid to flow within a predetermined time window.

12. The device of any one of the preceding claims, wherein the analysis area comprises a visible mark, the visible mark indicating an expected distance for the bodily fluid to flow within a predetermined time window.

13. The device of any one of the preceding claims, wherein the rheological property is viscosity.

14. The device of any one of the preceding claims, wherein the rheological property is shear rate or shear stress.

15. The device of claim 1, wherein the first surface of the housing further comprises a mark adjacent the analysis opening, the mark indicating an expected distance for the bodily fluid to flow within a predetermined time window.

16. The device of claim 1, wherein the analysis area comprises a visible mark, the visible mark indicating an expected distance for the bodily fluid to flow within a predetermined time window.

17. The device of claim 1, wherein the rheological property is viscosity.

18. The device claim 1, wherein the rheological property is shear rate or shear stress.

19. A method of diagnosing a disease state by evaluating one or more rheological properties of a bodily fluid, the method comprising:
providing a sample of bodily fluid to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area,
comparing an observable rheological property of the bodily fluid with an expected rheological property associated with the bodily fluid in a healthy state, the observable rheological property comprising the flow of the bodily fluid from the inlet area toward the analysis area, and
diagnosing a disease state if the observable rheological property does not meet the expected rheological property,
wherein the lateral flow strip comprises a reducing buffer solution.

20. The method of claim 19, wherein the disease state is selected from one of the following: sickle cell disease, dyslipidemia, coagulopathy, venous thrombosis, hemoglobinopathy, thalassemia, and compound heterozygous sickle cell diseases.

21. The method of claim 19, wherein the disease state is selected from one of the following: hyper IgM syndrome, Waldenstrom macroglobulinemia, primary amyloidosis, multiple myeloma, chronic lymphocytic leukemia, polycythemia, and cryoglobulinemia.

22. The method of any one of claims 19 through 21, wherein the expected rheological property is a minimum flow rate of the bodily fluid across the analysis area.

23. The method of any one of claims 19 through 22, further comprising providing a housing in which the lateral flow strip is disposed, the housing defining an inlet opening and an analysis opening on a first outer surface thereof, the inlet opening being adjacent the inlet area, and the analysis opening being adjacent at least a portion of the analysis area.

24. The method of claim 23, wherein the lateral flow strip comprises a first lateral flow strip, and the method further comprises removing the first lateral flow strip from the housing and inserting a second lateral flow strip into the housing.

25. The method of claim 24, further comprising:
providing a calibration solution associated with the disease state, the calibration solution having the expected rheological property of the bodily fluid in the healthy state,
depositing the calibration solution onto the input area of the first or second lateral flow strip, and
identifying the expected rheological property for the bodily fluid based on rheological property of the calibration solution.

26. The method of any one of claims 19 through 24, the method further comprising providing a calibration solution associated with the disease state, the calibration solution having the expected rheological property of the bodily fluid in the healthy state.

27. The method of claim 19, further comprising providing a housing in which the lateral flow strip is disposed, the housing defining an inlet opening and an analysis opening on a first outer surface thereof, the inlet opening being adjacent the inlet area, and the analysis opening being adjacent at least a portion of the analysis area.

28. The method of claim 19, wherein the lateral flow strip comprises a first lateral flow strip, and the method further comprises removing the first lateral flow strip from the housing and inserting a second lateral flow strip into the housing.

29. The method of claim 28, further comprising:
providing a calibration solution associated with the disease state, the calibration solution having the expected rheological property of the bodily fluid in the healthy state,
depositing the calibration solution onto the input area of the first or second lateral flow strip, and
identifying the expected rheological property for the bodily fluid based on rheological property of the calibration solution.

30. The method of claim 19, the method further comprising providing a calibration solution associated with the disease state, the calibration solution having the expected rheological property of the bodily fluid in the healthy state.

31. A test kit for monitoring a disease state, the disease state altering one or more rheological properties of a bodily fluid, the test kit comprising:
a testing device comprising:
a housing defining a hollow interior portion, the housing comprising a first end and a second end spaced apart from and opposite the first end, the housing defining an inlet opening, the inlet opening being adjacent the first end of the housing; and
a lateral flow strip disposable within the hollow interior portion, the lateral flow strip having a first end and a second end, the first and second ends of the lateral flow strip being opposite and spaced apart from each other, wherein the lateral flow strip comprises an inlet area adjacent the first end of the lateral flow strip, an absorbent area adjacent the second end of the lateral flow strip, and an analysis area disposed between the inlet area and the absorbent area, the inlet area being disposable below the inlet opening for receiving bodily fluid through the inlet opening,
wherein:
the lateral flow strip comprises a reducing buffer solution,
the housing defines an analysis opening through which at least a portion of the analysis area of the lateral flow strip is visible,
the bodily fluid has an expected rheological property across the analysis area that is associated with a healthy state, and
an observable rheological property of the bodily fluid is comparable to the expected rheological property to identify whether a disease state is present.

32. The test kit of claim 31, further comprising an applicator for receiving the bodily fluid from a patient and dispensing the bodily fluid on the inlet area.

33. The test kit of claim 32, wherein the applicator is a pipette.

34. The test kit of any one of claims 31 through 33, wherein the lateral flow strip is a first lateral flow strip and is removable from the housing, the kit further comprising a second lateral flow strip that is disposable within and removable from the housing.

35. The test kit of any one of claims 31 through 34, further comprising a calibration solution associated with the disease state, the calibration solution having the expected rheological property.

36. The test kit of any one of claims 31 through 35, wherein the reducing buffer solution comprises an inorganic reducing salt.

37. The test kit of any one of claims 31 through 35, wherein the reducing buffer solution is mixed with the bodily fluid.

38. The test kit of any one of claims 31 through 37, wherein one of the analysis area or the housing adjacent the analysis opening comprises a visible mark, the visible mark indicating an expected distance for the bodily fluid to flow within a predetermined time window.

39. The test kit of claim 31, further comprising a calibration solution associated with the disease state, the calibration solution having the expected rheological property.

40. The test kit of claim 31, wherein the reducing buffer solution comprises an inorganic reducing salt.

41. The test kit of claim 31, wherein the reducing buffer solution is mixed with the bodily fluid.

42. The test kit of claim 31, wherein one of the analysis area or the housing adjacent the analysis opening comprises a visible mark, the visible mark indicating an expected distance for the bodily fluid to flow within a predetermined time window.

43. A method of diagnosing a blood disorder disease, the method comprising:
providing a sample of blood to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area;
capturing, via an image sensor, an image of the analysis area of the lateral flow strip within a field of view of the image sensor and electrically communicating image data associated with the captured image to a computer processor;
calculating, with the computer processor, a signal to noise ratio (SNR) based on the image data, the computer processor being in electrical communication with a memory, the memory storing instructions executable by the computer processor; and
identifying, by the computer processor, a biomarker density associated with the calculated SNR.

44. The method of claim 43, wherein the image sensor, the computer processor, and the memory are disposed within a mobile computing device.

45. The method of claim 43, wherein the image sensor is coupled to a mobile computing device, and the computer processor and memory are remotely disposed from the mobile computing device.

46. The method of any one of claims 43-45, wherein the image data is communicated to the computer processor on a frame by frame basis.

47. The method of any one of claims 43-46, wherein the image sensor is a two dimensional camera.

48. The method of any one of claims 43-46, wherein the image sensor is a three dimensional camera.

49. The method of any one of claims 43-48, wherein the biomarker density comprises a percentage of the biomarker in the blood sample.

50. The method of any one of claims 43-49, wherein the blood disorder disease is sickle cell disease, and the biomarker density is a hemoglobin S (Hb S) density.

51. The method of claim 43, wherein the image sensor is a two dimensional camera.

52. The method of claim 43, wherein the image sensor is a three dimensional camera.

53. The method of claim 43, wherein the biomarker density comprises a percentage of the biomarker in the blood sample.

54. The method of claim 43, wherein the blood disorder disease is sickle cell disease, and the biomarker density is a hemoglobin S (Hb S) density.

55. A system for diagnosing a blood disorder disease by evaluating one or more rheological properties of a sample of blood, the system comprising:
a lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area, wherein an inlet area of the lateral flow strip is configured for receiving the blood sample;
an image sensor for capturing an image of the analysis area of the lateral flow strip;
a computer processor in electrical communication with the image sensor and a memory, the memory storing instructions executable by the processor that cause the processor to:
receive image data associated with the image captured by the image sensor;
calculate a signal to noise ratio (SNR) from the image data; and
identify a biomarker density associated with the calculated SNR.

56. The system of claim 55, wherein the instructions further cause the processor to communicate the biomarker density to a health care worker.

57. The system of claim 55, wherein the image sensor, the computer processor, and the memory are disposed within a mobile computing device.

58. The system of claim 55, wherein the image sensor is coupled to a mobile computing device, and the computer processor and memory are remotely disposed from the mobile computing device.

59. The system of any one of claims 55-58, wherein the image data is communicated to the computer processor on a frame by frame basis.

60. The system of any one of claims 55-59, wherein the image sensor is a two dimensional camera.

61. The system of any one of claims 55-59, wherein the image sensor is a three dimensional camera.

62. The system of any one of claims 55-61, wherein the biomarker density comprises a percentage of the biomarker in the blood sample.

63. The system of any one of claims 55-52, wherein the blood disorder disease is sickle cell disease, and the biomarker density is a hemoglobin S (Hb 5) density.

64. The system of claim 55, wherein the image sensor is a two dimensional camera.

65. The system of claim 55, wherein the image sensor is a three dimensional camera.

66. The system of claim 55, wherein the biomarker density comprises a percentage of the biomarker in the blood sample.

67. The system of claim 55, wherein the blood disorder disease is sickle cell disease, and the biomarker density is a hemoglobin S (Hb S) density.

68. A method of displaying a bodily fluid in a rheological property context, the method comprising:
depositing a sample of bodily fluid to an inlet area of a lateral flow strip, the lateral flow strip having a first end and a second end, the first and second ends of the lateral flow strip being opposite and spaced apart from each other, the inlet area being adjacent the first end of the lateral flow strip, and the lateral flow strip further comprising an absorbent area adjacent the second end of the lateral flow strip and an analysis area disposed between the inlet area and the absorbent area; and
allowing the bodily fluid to migrate through the lateral flow strip from the inlet area towards the analysis area for a defined period of time, wherein the defined period of time is the time a reference fluid having a rheological property takes to migrate from the inlet area to a point in the analysis area, and wherein the location of the bodily fluid relative to the analysis area after the defined period of time displays the bodily fluid in a rheological property context.
